(19) United States
(12) Patent Application Publication
(10) Pub. No.: US 2014/0189944 A1
(43) Pub. Date: Jul. 10, 2014

(54) GOGGLE APPARATUS

(71) Applicants: Kelly Lynd, Brentwood, TN (US); Nicholas Joseph Vallo, New Albany, OH (US); Kathryn Anne Carlson, Columbus, OH (US)

(72) Inventors: Kelly Lynd, Brentwood, TN (US); Nicholas Joseph Vallo, New Albany, OH (US); Kathryn Anne Carlson, Columbus, OH (US)

(21) Appl. No.: 14/146,754

(22) Filed: Jan. 3, 2014

Related U.S. Application Data
(60) Provisional application No. 61/749,284, filed on Jan. 5, 2013.

(51) Int. Cl. A61F 9/02; A61F 11/06

(52) U.S. Cl. CPC A61F 9/02; A61F 11/06
USPC 2/452

(57) ABSTRACT

Various embodiments of a goggle apparatus are disclosed. In one embodiment, a goggle apparatus with a frame, an eye covering, and one or more straps for retaining the goggle apparatus to the head of a user is provided, wherein the one or more straps is configured to cover at least a portion of a user's ear(s).

### CROSS-REFERENCE TO RELATED APPLICATIONS

This application claims priority from U.S. Provisional Patent Application No. 61/749,284, filed on Jan. 5, 2013, which is incorporated by reference herein in its entirety.

### BACKGROUND

Various sports and activities include the use of protective goggles to cover and protect a participant's eyes. Examples of such sports and activities include swimming, skiing, motorcycle riding, and working in hazardous environments.

Additionally, various sports and activities may include the use of ear coverings, which may serve, for example, to prevent items from leaving a user's ears, prevent materials from entering a user's ears, protect a user's hearing, present a profile having less drag, and keep a user's ears warm.

Many sports and activities that include the use of protective goggles also include the use of ear coverings. What is needed is a combined goggle apparatus and ear coverings.

### SUMMARY

In one embodiment, a goggle apparatus is provided, the apparatus comprising: a frame; at least one eye covering connected to the frame and comprising a transparent lens; an upper strap comprising an upper strap first end connected to the frame at a first point and an upper strap second end connected to the frame at a second point; and a lower strap comprising a lower strap first end connected to the upper strap at a first point and a lower strap second end connected to the upper strap at a second point.

In another embodiment, a lower strap apparatus is provided, the apparatus comprising: a lower strap first end; and a lower strap second end; wherein the lower strap first end is configured to connect to a goggle strap at a first point; wherein the lower strap second end is configured to connect to a goggle strap at a second point; and wherein the lower strap is configured to cover at least a portion of a user's ear.

In another embodiment, a goggle apparatus is provided, the apparatus comprising: a frame; at least one eye covering connected to the frame and comprising a transparent lens; and a strap comprising an upper portion and a lower portion, wherein the upper portion and the lower portion are both connected to the frame at a first point and a second point, and wherein the lower portion is configured to cover at least a portion of a user's ear.

### BRIEF DESCRIPTION OF THE DRAWINGS

The accompanying figures, which are incorporated in and constitute a part of the specification, illustrate various example apparatuses and systems, and are used merely to illustrate various example embodiments. In the figures, like elements bear like reference numerals.

FIG. 1 illustrates a side view of an example arrangement of a goggle apparatus.

FIG. 2 illustrates a side view of an example arrangement of a goggle apparatus.

FIG. 3a illustrates a perspective view of an example arrangement of a goggle apparatus.

FIG. 3b illustrates a rear view of an example arrangement of a goggle apparatus.

FIG. 4 illustrates a perspective view of an example arrangement of a goggle apparatus.

### DETAILED DESCRIPTION

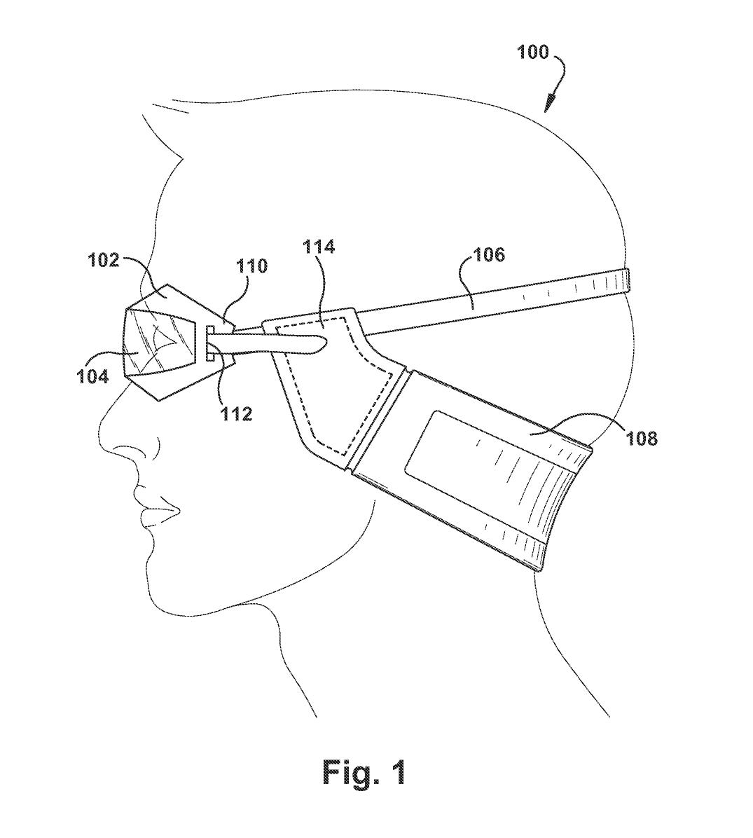

FIG. 1 illustrates an example arrangement of a goggle apparatus 100. Goggle apparatus 100 may comprise a frame 102 and at least one eye covering 104 connected to frame 102. Goggle apparatus 100 may also comprise an upper strap 106 and a lower strap 108.

Frame 102 may comprise a first point 110, to which an upper strap first end may be connected. Frame 102 may also comprise a second point (not shown) to which an upper strap second end may be connected. In one embodiment, frame 102 comprises a resilient material configured to conform to a user's face to create at least one of a water impermeable seal and an air impermeable seal. In one embodiment, frame 102 comprises any of a variety of materials, including for example, a polymer, a metal, and a composite.

At least one eye covering 104 may be connected to frame 102. In one embodiment, at least one eye covering 104 may be removably connected to frame 102. In one embodiment, at least one eye covering 104 may be connected to frame 102 so as to create at least one of a water impermeable seal and an air impermeable seal. In one embodiment, at least one eye covering 104 comprises a lens. In another embodiment, at least one eye covering 104 comprises a transparent lens. In another embodiment, at least one eye covering 104 comprises a protective shield configured to protect a user's eyes from water, wind, debris, impact, UV light, and other hazardous elements. In another embodiment, at least one eye covering 104 comprises a plurality of eye coverings, for example, one configured to substantially align with each of a user's eyes. In one embodiment, at least one eye covering 104 comprises any of a variety of materials, including for example, a polymer, a glass, a metal, and a composite.

Upper strap 106 may extend from frame 102 at a first point, about a user's head, and back to frame 102 at a second point. In one embodiment, upper strap 106 comprises an upper strap first connected to frame 102 at a first point 110 and upper strap second end connected to frame 102 at a second point (not shown).

In one embodiment, upper strap 106 comprises at least one adjustment mechanism 112. Adjustment mechanism 112 may comprise a slot, a clasp, or other devices selectively connecting upper strap 106 to frame 102 at first point 110 and second point (not shown). Adjustment mechanism 110 may be capable of retaining upper strap under tension. In another embodiment, upper strap comprises an adjustment mechanism including at least one tri-glide, clasp or other retention device positioned on upper strap 106 so as to selectively increase or decrease the length of upper strap 106.

Upper strap 106 may be configured to at least substantially extend about a user's parietal bone when a user is wearing goggle apparatus 100. In one embodiment, upper strap 106 comprises a resilient material. In another embodiment, upper strap 106 comprises any of a variety of materials, including for example, a polymer, a textile, and leather. In one embodiment, upper strap 106 comprises a width between about 0.1 in. and about 1.0 in. In another embodiment, upper strap 106 comprises a width between about 0.15 in. and about 0.5 in. In another embodiment, upper strap 106 comprises a width between about 0.2 in. and about 0.3 in.

Lower strap 108 may extend from frame 102 at a first point, about a user's head, and back to frame 102 at a second point. In one embodiment, lower strap 108 comprises a lower strap first end and a lower strap second end. The lower strap first end may be connected to upper strap 106 at a first point 114, while the lower strap second end may be connected to upper strap 106 at a second point (not shown). In one embodiment, lower strap 108 is connected to upper strap 106 by at least one of stitching, gluing, and ultrasonic welding. In another embodiment, lower strap 108 is slidably connected to upper strap 106. In another embodiment, lower strap 108 may be integrally formed with upper strap 106, in which embodiment lower strap first end and lower strap second end may comprise junction points between lower strap 108 and upper strap 106. In one embodiment, lower strap first end and lower strap second end are selectively connected to upper strap 106. In another embodiment, lower strap first end and lower strap second end are selectively connected to upper strap 106 such that a user may attach or separate lower strap 108 and upper strap 106 at the user's convenience.

In one embodiment, lower strap 108 is configured to be added to pre-existing goggles comprising a goggle strap 106. Lower strap 108 may comprise a lower strap first end and a lower strap second end. In one embodiment, the lower strap first end is configured to connect to goggle strap 106 at a first point, wherein the lower strap second end is configured to connect to goggle strap 106 at a second point.

In one embodiment, lower strap 108 is configured to cover at least a portion of a user's ear when installed on goggle strap 106. In another embodiment, lower strap 108 is configured to cover an external auditory canal of a user's ear.

In one embodiment, the lower strap first end and the lower strap second end may be connected to goggle strap 106 of pre-existing goggles. In another embodiment, the lower strap first end and the lower strap second end are configured to at least one of slidably connect, selectively connect, and integrally connect to goggle strap 106.

In one embodiment, lower strap 108 comprises at least one adjustment mechanism (not shown). The adjustment mechanism may comprise a tri-glide, a clasp, a hook and loop fastener, or any other retention device capable of adjusting the length and/or tension in lower strap 108. Lower strap 108 may be configured to at least substantially extend about a user's occipital bone when a user is wearing goggle apparatus 100.

In one embodiment, lower strap 108 is configured to cover at least a portion of a user's ear when a user is wearing goggle apparatus 100. In another embodiment, lower strap 108 is configured to cover an external auditory canal of a user's ear(s). In another embodiment, lower strap 108 is configured to cover the entirety of a user's ear(s). In one embodiment, lower strap 108 is configured to at least substantially maintain an ear plug in the external auditory canal of the user's ear(s). Goggle apparatus 100 may be utilized by a swimmer to keep water out of the swimmer's eye(s) while maintaining an ear plug in the external auditory canal of the swimmer's ear(s) so as to keep water out of the swimmer's ear(s).

In one embodiment, lower strap 108 is configured to keep water, air, debris, loud sounds, and other hazardous materials out of a user's ear(s). In another embodiment, lower strap 108 is configured to keep a user's ear(s) warm. In another embodiment, lower strap 108 comprises at least one heater to keep a user's ear(s) warm. In another embodiment, lower strap 108 comprises at least one speaker configured to direct sound into a user's ear(s), which speakers may be connected to an electronic device capable of transmitting sound, such as a two-way radio, AM/FM radio, satellite radio, or a portable MP3 player. In one embodiment, lower strap 108 may be configured to maintain a user's ear(s) close to the user's head so as to improve an aerodynamic or hydrodynamic profile of the user. In one embodiment, lower strap 108 may be interchangeable. In another embodiment, lower strap 108 may comprise any of various colors and may be coordinated with a team's colors. In another embodiment, lower strap 108 may comprise a display surface for displaying a team or sponsor's name or logo on lower strap 108.

Lower strap 108 may comprise a width between about 0.75 in. and about 2.25 in. In one embodiment, lower strap 108 comprises a width between about 1.0 in. and about 2.0 in. In another embodiment, lower strap 108 comprises a width between about 1.25 in. and about 1.75 in. Lower strap 108 may comprise a resilient material. In one embodiment, lower strap 108 comprises neoprene. In another embodiment, lower strap 108 comprises any of a variety of materials, including for example, a polymer, a textile, and leather.

Goggle apparatus 100 may in one form be used as swimming goggles configured to keep ear plugs in a user's ears, so as to keep water out of both a user's ears and eyes. In another embodiment, goggle apparatus 100 may include ski goggles configured to keep a user's ears warm. In another embodiment, goggle apparatus 100 may include motorcycle goggles configured to keep a user's ears warm or configured to allow a user to listen to audio. In another embodiment, goggle apparatus 100 may include any safety goggles to keep a user's eyes and ears shielded from any hazardous environment.

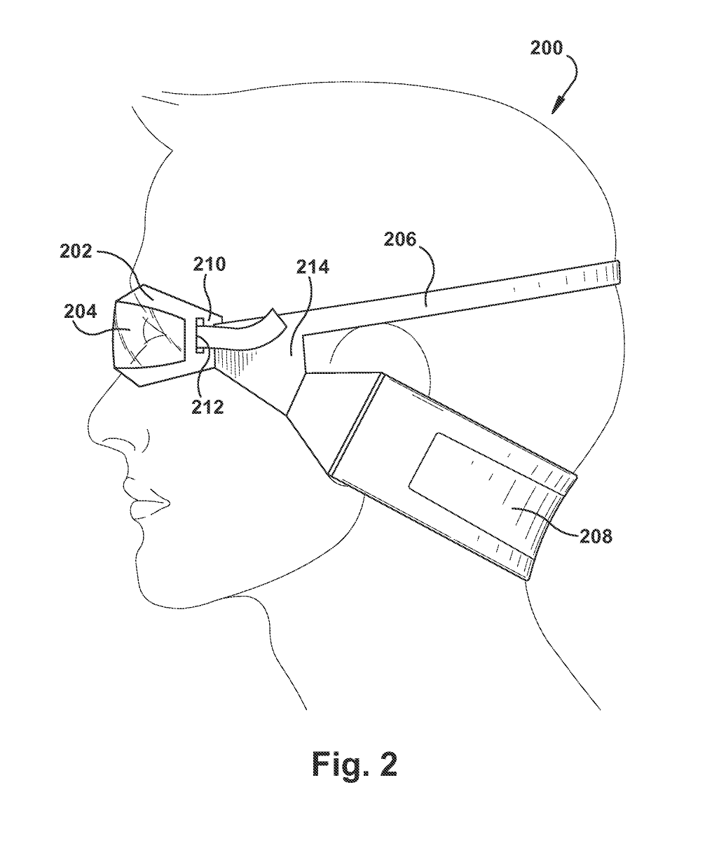

FIG. 2 illustrates an example arrangement of a goggle apparatus 200. Goggle apparatus 200 may comprise a frame 202 and at least one eye covering 204 connected to frame 202. Goggle apparatus 200 may also comprise an upper strap 206 and a lower strap 208. Upper strap 206 may be connected to frame 202 at a first point 210 and a second point (not shown). Upper strap 206 may comprise an adjustment mechanism 212 configured to allow an adjustment in the length and tension of upper strap 206. Lower strap 208 may be integrally connected to upper strap 206 at a first point 214 and a second point (not shown).

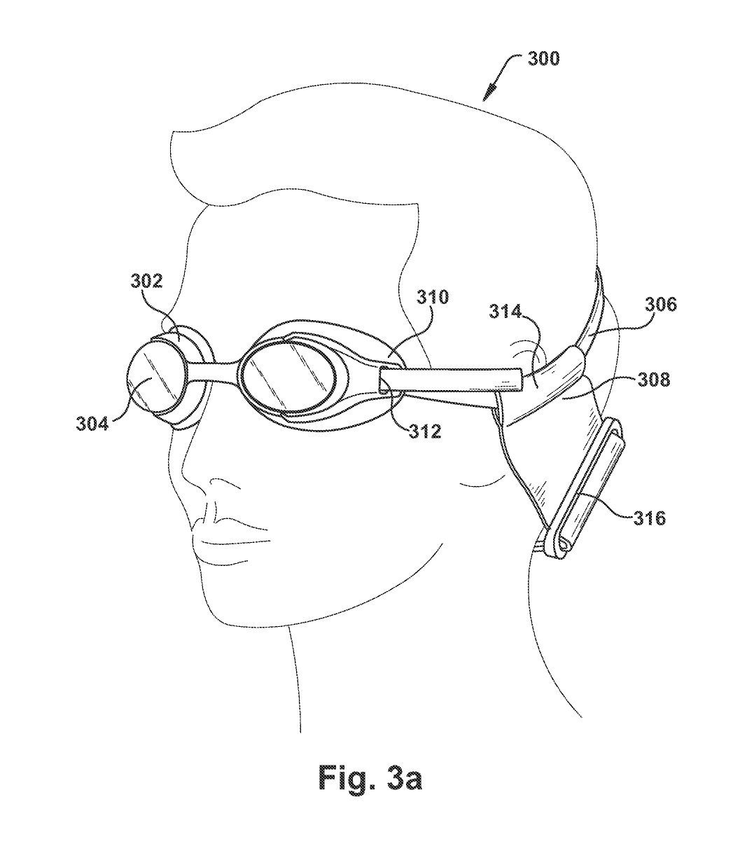

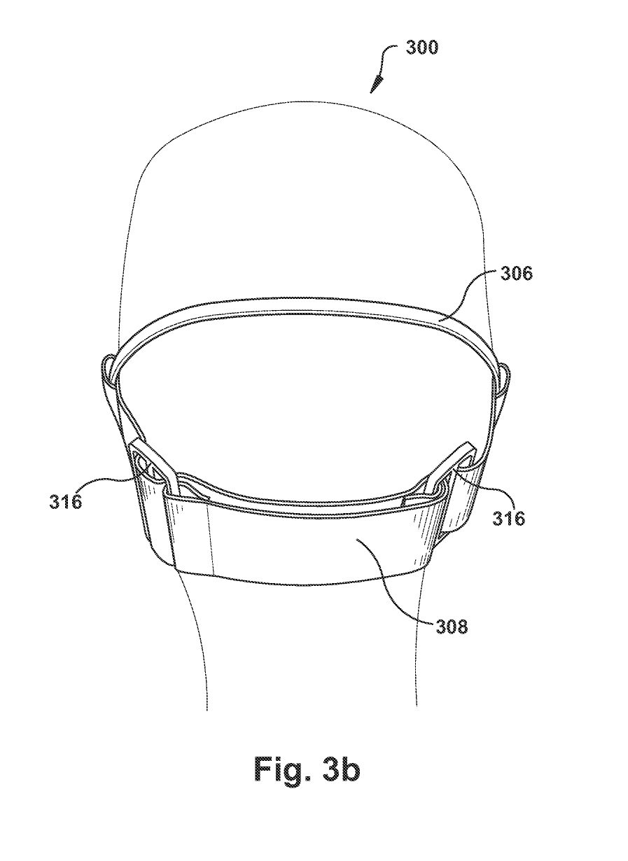

FIG. 3a illustrates an example arrangement of a goggle apparatus 300. Goggle apparatus 300 may comprise a frame 302 and at least one eye covering 304 connected to frame 302. Goggle apparatus 300 may also comprise an upper strap 306 and a lower strap 308. Upper strap 306 may be connected to frame 302 at a first point 310 and a second point (not shown). Upper strap 306 may comprise an adjustment mechanism 312 configured to allow an adjustment in the length and tension of upper strap 306. Lower strap 308 may be connected to upper strap 306 at a first point 314 and a second point (not shown). Lower strap 308 may comprise at least one adjustment mechanism 316 configured to allow an adjustment in the length and tension of lower strap 308.

FIG. 3b illustrates another view of an example arrangement of a goggle apparatus 300. Lower strap 308 may comprise a plurality of adjustment mechanisms 316 configured to allow an adjustment in the length and tension of lower strap 308. In one embodiment, adjustment mechanism 316 comprises a tri-glide mechanism configured to allow a user to selectively set the length and tension of lower strap 308. In another embodiment, adjustment mechanism 316 may comprise one or more of any clasp, clamp, or other retention mechanism capable of maintaining a desired length and tension in lower strap 308.

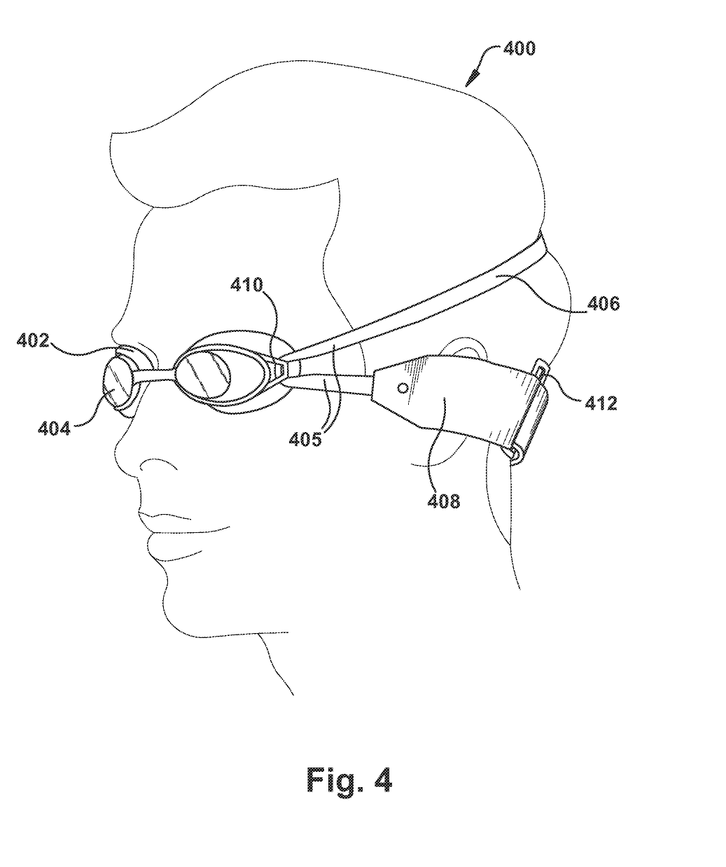

FIG. 4 illustrates an example arrangement of a goggle apparatus 400. Goggle apparatus 400 may comprise a frame 402 and at least one eye covering 404 connected to frame 402. Goggle apparatus 400 may also comprise a strap 405 comprising an upper portion 406 and a lower portion 408.

In one embodiment, upper portion 406 and lower portion 408 are each connected to frame 402 at a first point 410 and a second point (not shown). In one embodiment, strap 405 extends through a slot at first point 410 and a second point (not shown) and is allowed to slide about its length within the slot to accommodate adjustment of strap 405.

Upper portion 406 may be configured to extend about a user's parietal bone when a user is wearing goggle apparatus 400. In one embodiment, upper portion 406 comprises an attachment mechanism configured to allow a user to selectively separate upper portion 406 from itself so that strap 405 may be installed at first point 410 and a second point (not shown). The attachment mechanism may comprise any mechanism capable of selectively attaching two sections of upper portion 406 to one another, including for example, a clip, a clasp, a snap, and a tri-glide. In another embodiment, upper portion 406 comprises an adjustment mechanism configured to allow a user to increase the length and/or tension of upper portion 406.

Lower portion 408 may be configured to extend about a user's occipital bone when a user is wearing goggle apparatus 400. In one embodiment, lower portion 408 comprises at least one adjustment mechanism 412 configured to allow a user to increase the length and/or tension of lower portion 408. Lower portion 408 may be configured to cover at least a portion of a user's ear(s). In one embodiment, lower portion 408 is configured to cover an external auditory canal of a user's ear(s). In another embodiment, lower portion 408 is configured to maintain an ear plug in the external auditory canal of a user's ear(s).

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See Bryan A. Garner, A Dictionary of Modern Legal Usage 624 (2d. Ed. 1995). Also, to the extent that the terms “in” or “into” are used in the specification or the claims, it is intended to additionally mean “on” or “onto.” To the extent that the term “selectively” is used in the specification or the claims, it is intended to refer to a condition of a component wherein a user of the apparatus may activate or deactivate the feature or function of the component as is necessary or desired in use of the apparatus. To the extent that the term “operatively connected” is used in the specification or the claims, it is intended to mean that the identified components are connected in a way to perform a designated function. As used in the specification and the claims, the singular forms “a,” “an,” and “the” include the plural. Finally, where the term “about” is used in conjunction with a number, it is intended to include ±10% of the number. In other words, “about 10” may mean from 9 to 11.

As stated above, while the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art, having the benefit of the present application. Therefore, the application, in its broader aspects, is not limited to the specific details, illustrative examples shown, or any apparatus referred to. Departures may be made from such details, examples, and apparatuses without departing from the spirit or scope of the general inventive concept.

1. A goggle apparatus comprising:
a frame;
at least one eye covering connected to the frame and comprising a transparent lens;
an upper strap comprising an upper strap first end connected to the frame at a first point and an upper strap second end connected to the frame at a second point; and
a lower strap comprising a lower strap first end connected to the upper strap at a first point and a lower strap second end connected to the upper strap at a second point.

2. The apparatus of claim 1, wherein the lower strap is configured to cover at least a portion of a user's ear.

3. The apparatus of claim 1, wherein the lower strap is configured to cover an external auditory canal of a user's ear.

4. The apparatus of claim 1, wherein the upper strap comprises at least one adjustment mechanism.

5. The apparatus of claim 1, wherein the lower strap comprises at least one adjustment mechanism.

6. The apparatus of claim 1, wherein the lower strap first end and the lower strap second end are at least one of slidably connected, selectively connected, and integrally connected to the upper strap.

7. The apparatus of claim 1, wherein the lower strap comprises a width between about 0.75 in. and about 2.25 in.

8. The apparatus of claim 1, wherein the lower strap is configured to at least substantially extend about a user's occipital bone, and wherein the upper strap is configured to at least substantially extend about a user's parietal bone.

9. The apparatus of claim 1, wherein the lower portion comprises a neoprene material.

10. The apparatus of claim 1, wherein the lower strap is configured to at least substantially maintain an ear plug in the external auditory canal of the user's ear.

11. A lower strap apparatus comprising:
a lower strap first end; and
a lower strap second end;
wherein the lower strap first end is configured to connect to a goggle strap at a first point;
wherein the lower strap second end is configured to connect to a goggle strap at a second point; and
wherein the lower strap is configured to cover at least a portion of a user's ear.

12. The apparatus of claim 11, wherein the lower strap is configured to cover an external auditory canal of a user's ear.

13. The apparatus of claim 11, wherein the lower strap first end and the lower strap second end are configured to at least one of slidably connect, selectively connect, and integrally connect to the goggle strap.

14. A goggle apparatus comprising:
a frame;
at least one eye covering connected to the frame and comprising a transparent lens; and
a strap comprising an upper portion and a lower portion,
wherein the upper portion and the lower portion are both connected to the frame at a first point and a second point, and
wherein the lower portion is configured to cover at least a portion of a user's ear.

15. The apparatus of claim 14, wherein the lower portion is configured to cover an external auditory canal of a user's ear.

16. The apparatus of claim 14, wherein the lower portion comprises at least one adjustment mechanism.

17. The apparatus of claim 14, wherein the upper portion comprises at least one attachment mechanism.

18. The apparatus of claim 14, wherein the lower portion comprises a width between about 0.75 in. and about 2.25 in.

19. The apparatus of claim 14, wherein the lower portion is configured to at least substantially extend about a user's occipital bone and wherein the upper portion is configured to at least substantially extend about a user's parietal bone.

20. The apparatus of claim 14, wherein the lower portion comprises a neoprene material.
